(19) United States
(12) Patent Application Publication
(10) Pub. No.: US 2004/0146520 A1
(43) Pub. Date: Jul. 29, 2004

(54) Materials and methods relating to immune suppression

(76) Inventors: Gavin Screaton, Oxford (GB); Xiaoning Xu, Oxford (GB)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/472,328

(22) PCT Filed: Mar. 19, 2002
(86) PCT No.: PCT/GB02/01337

(30) Foreign Application Priority Data
Mar. 19, 2001 (GB) 0106772.7
May 14, 2001 (GB) 011781.1

(51) Int. Cl. A61K 39/00

(52) U.S. Cl. 424/185.1

(57) ABSTRACT

The invention provides immunocomplex comprising MHC molecules or functional fragments thereof which are modified so as to prevent binding to co-receptors e.g. CD8 or CD4. The inventors have determined that inability of the MHC complex to bind co-receptor leads to death of the T-cells without delivery of an activation/proliferation signal. By associating the immunocomplex with a specific peptide antigen it is possible to selectively suppress the immune system of a host, i.e. to help prevent tissue rejection or treat autoimmune diseases. For a more universal suppression of the immune system, it is possible to administer the modified MHC complex or fragment/component thereof in the absence of peptide antigen. For example, modified βM complex can be administered.

### FIELD OF THE INVENTION

The present invention concerns materials and methods relating to selective immune suppression. Particularly, but not exclusively, the present invention relates to methods of substantially reducing an immune response by the specific killing of T lymphocytes. The invention further relates to the use of this method as a medical treatment, particularly with regard to autoimmune diseases and as a prevention of tissue transplantation rejection.

### BACKGROUND OF THE INVENTION

The engagement of T-cell receptor (TCR) with peptide-MHC complexes is a critical event in the initiation of the T cell response. Binding of the co-receptors CD4 or CD8 to the MHC plays an important role to augment TCR-triggered activation of most T cells. Signalling via the TCR can have a number of outcomes, ranging from activation/proliferation through anergy to apoptosis [Van Parijs, 1998; Monks, 1998].

After TCR engagement, CTL kill target cells by two major pathways. Perforin-dependent cytotoxicity involves the exocytosis of pre-formed CTL granules containing perforin and various granzymes, which synergise to induce death of the target cells. Activated CTL also express Fas ligand (FasL) which can engage Fas on the target cells and trigger apoptosis through a well characterised pathway. Upregulation of FasL on CTL is a two edged sword as it also exposes CTL, which express Fas, to the risk of self induced death. Activation induced cell death (AICD) is an important immunological control mechanism and can occur shortly after activation where it is mainly triggered by Fas/FasL interactions. AICD plays an important role in peripheral tolerance and mice or humans with mutations in the Fas or FasL genes develop a lymphoproliferative syndrome consisting of lymphadenopathy, splenomegaly, hyper-gammaglobulinaemia and a variety of autoimmune manifestations. [Fisher, 1995].

Selective deletion of autoreactive T cells is a major goal for the treatment of autoimmune disease or to prevent the rejection of transplanted organs. Although effective in many cases, conventional therapy relies upon broad spectrum immunosuppression with the consequent risk of opportunistic infection or tumorogenesis. One route for targetted immunotherapy is the systemic administration of agonist peptide to induce apoptosis of reactive lymphocytes. This approach has two major limitations: firstly in many cases the responsible peptide epitopes have not yet been identified. Secondly, although such therapy has been shown to work in animal models, the activation, proliferation and cytokine release which is induced as a consequence of activation can lead to damage of the lymphoid organs and general immunosuppression [Aichele, 1997].

Thus, the present inventors have appreciated that there exists a need for an effective mechanism for selectively deleting autoreactive T cells without causing general immunosuppression. Further, the inventors have realised that it would be advantageous to develop such a mechanism that was not reliant on knowledge of the peptide epitopes as, as mentioned above, not all responsible peptide epitopes have yet been identified.

### SUMMARY OF THE INVENTION

The massive diversity of the T cell receptor gives T cells a very high level of specificity in target recognition. Autoimmune and allergic diseases are believed to involve inappropriate activation of the immune system and in some cases cytotoxic T cells (CTL). These T cells are likely to show peptide specificity although in many cases the exact antigens are yet to be defined. Likewise, the rejection of allogenic tissue grafts is also believed in large part to involve specific recognition by T cells.

The inventors have developed a novel approach to selectively delete antigen-specific CTL with minimal activation. They tested a number of human CTL clones in vitro or ex-vivo stimulated by MHC/peptide presented either by cells or as soluble multimeric complexes. The studies have revealed a surprising difference in the activation threshold for CTL killing of targets versus CTL apoptosis in the presence or absence of CD8/MHC interaction.

The use of MHC class I/peptide complexes which have been mutated so that the class I molecule no longer binds to CD8 allows efficient killing of the CTL without delivering an activation/proliferation signal. In this way, CTL can be deleted using these complexes in the absence of activation and proliferation which are likely to have unwanted side effects. In addition, the approach should prevent damage to tissues which occurs using potential peptide therapy because the peptide can bind to bystander cells turning them into yet more targets for the autoreactive CTL.

Thus, at its most general, the present invention relates to materials and methods concerned with the selective prevention or reduction of immune responses involving T cells, in particular cytotoxic T lymphocytes, e.g. in autoimmune diseases or as a response to a transplanted tissue.

In a first aspect of the invention, there is provided an immunocomplex comprising an MHC molecule or functional derivative or fragment thereof and a peptide antigen associated with said MHC, wherein said MHC molecule or functional derivative or fragment thereof has a modified co-receptor binding domain such that co-receptor interaction is prevented.

The prevention of co-receptor interaction is determined by the reduction in full T-cell activation. In other words, the pathways normally activated by the interaction between the co-receptor and MHC via the co-receptor binding domain are affected and in most cases prevented.

The immunocomplex may simply comprise a soluble complex of a modified MHC molecule (e.g. class I or class II, preferably class I) and a peptide antigen. However, the immunocomplex may be associated with a cell and be displayed on the cell membrane. The term “functional derivative or fragment thereof” includes the use of a molecule which is derived from a MHC molecule and which maintains the ability to display peptide antigen to a T cell. For example, it is possible to use β2 microglobulin of MHC class I molecules which has been mutated in the co-receptor binding domain so that co-receptor interaction is prevented.

When the MHC molecule is a class I molecule, the co-receptor binding domain will be the region that binds the class I co-receptor (CD8). When the MHC molecule is a class II molecule, the co-receptor binding domain will be the region that binds the CTL class II co-receptor (CD4). Work has previously been carried out to analyse the exact binding domains of MHC class I and class II molecules for CD8 and CD4 respectively (Gao, 1997; König, K. et al Nature Vol. 356 P 796 (1992); Cammarota G. et al Nature Vol. 356, P799 (1992); Gao, G. and Jakobsen, B. Review Immunology Today, vol. 21, No. 12 630 (2000)). For MHC class I molecules, the co-receptor (CD8) binding domain has been located to the α3 domain on the heavy chain and therefore it is preferred that this region is modified to prevent CD8 interaction with the immunocomplex in accordance with the invention. However, it may also be possible to mutate the α2 domain in order to prevent co-receptor interaction (Sun. J. Exp. Med. 182, 1275-1280, 1995). With regard to MHC class II molecules, the co-receptor (CD4) binding domain has been located to the β-chain 2 domain which is structurally analogous to the CD8 binding loop in MHC class I α3 domain. However, it may also be possible to mutate the α chain of class II MCH molecules in order to prevent co-receptor interaction (Konig. J. Exp. Med. 182, 779-787, 1995).

The person skilled in the art will be able to imagine many ways of modifying a ligand (e.g. α3 domain) such that the receptor (CD8) is prevented from binding. Such techniques include the use of blocking antibodies, blocking peptides and/or the use of small molecules or mimetics.

The invention further provides a method of identifying a substance capable of inhibiting binding between the MHC or β2M ligands and co-receptor (CD8 or CD4), said method comprising contacting said substance with said ligand and co-receptor in an environment where ligand and co-receptor would bind in the absence of said substance; and determining the binding between the ligand and the receptor. The method may further comprise the preparation of a medicament comprising said substance for use in suppressing the immune system in a host.

However, the inventors believe that greater specificity of blocking co-receptor binding is required to allow the present invention to be so advantageously selective. Therefore, the preferred modification is mutagenesis of the co-receptor binding domain (e.g. α3 domain or β2 domain for class I and class II molecules respectively) by either addition, substitution, or deletion of one or more amino acids native to this domain. Even more preferred is the substitution of one or more native amino acids with different (non-native) amino acids. The loop in α3 domain of class I is important for the CD8 binding and it contains about 30 amino acids from 220-250 of class I heavy chain. However, in which ever domain is chosen to be mutated, it is preferable that any one or combination of amino acids may be modified as described above so as to prevent the co-receptor interaction. In a preferred embodiment of the invention it is preferred that at least 2, or at least 3, or at least 3 to 10 amino acids are modified. In a even more preferred embodiment 2 amino acids are modified. In α3 domain these are preferably amino acids D and T at position 227 and 228 respectively. These amino acids are replaced by amino acids K and A respectively. In the α2 domain it is preferable to modify amino acids Gly 115, Asp 112 and/or Glu 128. Where β2M of MHC class I is used, it is preferable to modify amino acids at 58-60, particularly lysine 58 as this makes contact with an arginine in CD8 in both the human and mouse crystals. Modification of the amino acid sequence is preferably achieved at the nucleic acid level using standard well-known techniques.

As can be seen from above, the immunocomplexes of the present invention can be conveniently used to selectively suppress the immune system, based on the particular peptide antigen they are displaying.

However, there may well be situations where a more universal or general immune suppression in a host is desirable. With this in mind, the inventors have determined that the β2M component of MHC when modified to prevent co-receptor binding is still capable of folding correctly in the absence of MHC heavy chain. Thus, this component is a good example of an immunocomplex which could be used in the absence of peptide antigen to provide a general immune suppression in a host. This aspect of the invention is discussed below.

As seen above, the invention is preferably applied to MHC class I and MHC class II molecules and may be used in the selective suppression of the immune system owing to the interaction of the MHC with T cells. However, for convenience, where explanation of the invention is required, the text concentrates on the situation were the MHC is class I, the co-receptor is CD8 and the T cells are CTLs.

It is envisaged that the MHC molecule will be the allele or variant associated with the particular disease to be treated. For example, if the disease e.g. autoimmune disease, is associated with an HLA-A2 MHC class I molecule, then it would be desirable to suppress those CTL's restricted to this particular MHC allele. Therefore, it is preferable to use the particular MHC allele associated with the disease in question. Of course, the invention may also be used to suppress the CTL response to a transplanted tissue. In this case, the MHC molecule is preferably the MHC allele expressed by the cells of the donor tissue.

In a second aspect of the present invention, there is provided a method of producing immunocomplexes according to the invention suitable for treating a patient requiring selective immune suppression, said method comprising the steps of

(a) obtaining donor cells associated with eliciting an immune response requiring suppression;

(b) determining the MHC allele(s) expressed by those donor cells;

(c) manipulating said donor cells to express a modified variant of said MHC allele(s), said modified variant lacking the ability to bind T cell co-receptor (e.g. CD8 or CD4) but maintaining the ability to display peptide antigen as an immunocomplex;

(d) isolating said immunocomplex from the donor cells.

The donor cells associated with eliciting an immune response requiring suppression may be cells linked to an autoimmune disease, i.e. they are obtained from a patient suffering from the autoimmune disease, or they may be cells derived from a donor tissue prior to transplantation. The particular MHC allele expressed in these cells may be determined using standard and well known techniques e.g. by serology or PCR techniques to identify the particular HLA DNA.

Once the particular MHC allele is known, the cells may be manipulated to express a modified variant of the MHC. This may be achieved by mutating the wild type nucleic acid sequence of the MHC gene so that the expressed product is no longer capable of binding T cell co-receptors, e.g. CD8 or CD4 depending of the class of MHC molecule. Although less preferred, it may also be possible to block the expression of the MHC gene and replaced by nucleic acid (e.g. in the form of a vector transfected into the donor cell) which encodes for a modified MHC variant of the determined MHC allele.

As the modified MHC is being produced within the cells associated with eliciting the immune response requiring suppression, the modified MHC will complex with endogenous peptide antigens derived from those cells. Thus, an immunocomplex is produced that comprises a modified MHC molecule of the correct allotype associated with eliciting the immune response and which displays peptide antigens associated with those cells.

The method according to the second aspect of the invention may further comprise the step of purifying the immunocomplex for administration to a patient requiring selective immune suppression. The immunocomplex may further be used in the preparation of a medicament for selectively suppressing an immune response for, e.g. the treatment of autoimmune diseases or for administration to a recipient of a tissue transplant.

By producing the modified MHC molecule in the donor cells, the immunocomplex produced by the cell will carry the peptide antigens of that cell. This avoids the necessity to know the peptides associated with the immune response. However, where the peptide antigens are known, and the MHC type (e.g. allele) is either known or can be determined, it would be possible to produce immunocomplexes in accordance with the present invention by using cell lines transfected with a vector encoding a modified MHC molecule of the correct type (allele). The cell line would preferably be a laboratory based cell line that is already established. It would be preferable to chose a cell line that related to the tissue type associated with the autoimmune disease or the organ to be transplanted. For example, if the organ to be transplanted was a kidney then it would be preferable to use an established kidney cell line. The use of an established cell line avoids the requirement to produce a new cell line which can be time consuming. However, it may be preferable to try to establish a cell line from the cells associated with the autoimmune disease or the organ to be transplanted. A preferred cell line would be a B-cell line. Further, the cell line may be a MHC negative cell line but even if the cells were MHC positive, the inventors believe that the additional production of modified MHC will have a negative affect on native MHC.

The peptide antigen may be associated with the modified MHC molecule or functional fragment thereof by expressing a fusion protein comprising the β2M and the peptide in a cell also expressing a modified MHC heavy chain, e.g. modified in the α3 domain. The fusion protein and the heavy chain can then be folded to produce the immunocomplex in accordance with the present invention. Alternatively, the peptide could be associated with the β2M as a fusion protein and expressed in the absence of the MHC heavy chain. The β2M component (or functional fragment) of the MHC molecule is capable of folding correctly without the a heavy chain and thus can be used in accordance with the present invention with or without associated peptide. The β2M will be modified in the co-receptor binding domain as discussed herein.

The modified β2M carries a modification by either addition, substitution or deletion in its amino acid sequence particularly in the region of amino acid 57 to amino acid 61, more preferably amino acid 58 to amino acid 60. The modified β2M may have the sequence of any one of the mutants shown in Table I.

Each of the mutants provided in Table I form separate aspects of the invention. The preferred mutants are those that show inhibition of killing of target cells following a CTL assay (Xu et al. Immunity Vol 14 pages 591-602, 2001) see detailed description.

Particularly preferred β2M mutants include mutants comprising the modification of 60W to L; 60W to V and 59 deleted so 58k fused to 60W.

Thus, where selective suppression of the immune system of a host is desirable, the specific peptide antigen can be associated with the modified MHC complex or component (functional fragment) thereof, e.g. β2M. However, if a non-selective, i.e. universal immune suppression was required, it would be possible to administer an immunocomplex comprising simply the modified MHC or functional fragment thereof e.g. the modified β2M.

In a third aspect of the present invention, there is provided a method of producing an immunocomplex according to the invention suitable for use in treating a patient requiring selective immune suppression, said method comprising the steps of

(a) transfecting a cell (preferably an MHC negative cell) with a vector encoding a MHC molecule or functional derivative or fragment thereof, having a modified co-receptor binding domain such that co-receptor interaction (e.g. CD8 or CD4 receptors depending on the class of MHC molecule) is prevented;

(b) expressing said modified MHC molecule in said cell; and

(c) isolating and purifying said MHC molecule.

The immunocomplex may be isolated as a soluble complex or it may be isolated in associated with the cell upon which it is displayed.

If it is desirable that the immunocomplex displays a particular peptide antigen, this can be achieved by transfecting the cell with nucleic acid encoding the peptide antigen of interest prior to the expression of the MHC in the cell. Thus, the peptide antigen may be endogenous, in which case they will depend on the cell type used to produce the immunocomplex, or they can be chosen antigenic peptides which are introduced into the cell (usually at the nucleic acid level) at the time the MHC is expressed.

In a fourth aspect of the present invention, there is provided a method of treating a patient requiring selective immune suppression, said method comprising the steps of

(a) determining the MHC allele associated with eliciting the immune response requiring suppression;

(b) producing an immunocomplex comprising the MHC allele or functional derivative or fragment thereof and a peptide antigen; and

(c) administering said immunocomplex to said patient in order to selective suppress the immune response.

The patient requiring selective immune suppression may be one suffering from an autoimmune disease or it may be a recipient of a transplanted tissue.

In accordance with the present invention, there is provided a modified MHC molecule or functional fragment or derivative thereof, that is capable of immune suppression, or the selective suppression/reduction of the immune response to specific peptide.

The modified MHC molecule may be a modified MHC class I molecule that has been modified in either the α2 or α3 domain as described above. Alternatively, it may be the β2M component of the MHC class I molecule that has also been modified to prevent co-receptor binding. In each case, the modified molecules may be administered to suppress the immune system. Where specific or selective immune suppression is required, the modified molecules may be complexed to a peptide to form an immunocomplex again, as described above.

With regard to MHC class II, the α chain or the β-chain β2 domain, may be modified. These modified molecules may also be associated with a peptide to form an immunocomplex.

The immunocomplex or modified molecules may be administered to a patient as part of a medicament. Thus, the invention further comprises a pharmaceutical composition comprising an immunocomplex as described above. These compositions may comprise, in addition to the immunocomplex, a pharmaceutically acceptable excipient, carrier, buffer, stabiliser or other materials well known to those skilled in the art. Such materials should be reasonably non-toxic and should not interfere with the efficacy of the active ingredient. The precise nature of the carrier or other material may depend on the route of administration, e.g. oral, intravenous, cutaneous or subcutaneous, nasal, intramuscular, intraperitoneal routes.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Rather than administering the immunocomplex itself, it may be preferable to administer a patient with nucleic acid encoding the modified MHC. The nucleic acid can then express the modified MHC within the cells of the patient and produce an immunocomplex in accordance with the invention. The nucleic acid may be introduced into the patient's cells by means of gene therapy.

Vectors such as viral vectors have been used in the prior art to introduce genes into a wide variety of different target cells. Typically the vectors are exposed to the target cells so that transfection can take place in a sufficient proportion of the cells to provide a useful therapeutic or prophylactic effect from the expression of the modified MHC molecule. The transfected nucleic acid may be permanently incorporated into the genome of each of the targeted cells, providing long lasting effect, or alternatively the treatment may have to be repeated periodically.

A variety of vectors, both viral vectors and plasmid vectors, are known in the art, see U.S. Pat. No. 5,252,479 and WO 93/07282. In particular, a number of viruses have been used as gene transfer vectors, including papovaviruses, such as SV40, vaccinia virus, herpesviruses, including HSV and EBV, and retroviruses. Many gene therapy protocols in the prior art have used disabled murine retroviruses.

As an alternative to the use of viral vectors other known methods of introducing nucleic acid into cells includes electroporation, calcium phosphate co-precipitation, mechanical techniques such as microinjection, transfer mediated by liposomes and direct DNA uptake and receptor-mediated DNA transfer.

The inventors have found that although the cell will produce native MHC, the production of modified MHC within the same cell, has a negative effect on the native MHC. In other words, there is no requirement to prevent production of the native MHC. Rather modified MHC can simply be introduced into the cell and produced along side the native MHC and yet will be dominant. This means that the immunocomplex of the present invention may be administered in the form of protein or nucleic acid to expression protein (Gene therapy, DNA vaccines) and will still be active even in the presence of host MHC.

### BRIEF DESCRIPTION OF THE DRAWINGS

FIG. 1. Detection of Apoptosis of CTL and Target Cells.

(A) HIV-1 gag-specific A2-restricted CTL were co-cultured with an autologous BCL pre-pulsed with either gag index peptide (SLYNTVATL) or control peptide (HIV-1 pol). Cells were stained with anti-CD19-PE, anti-CD8-Tricolor, and Annexin-V-FITC. Apoptosis of CTL or targets was assessed by gating CD8 and CD19 positive populations respectively. (B) Gag-specific CTL were cocultured with the BCL's pre-pulsed with HIV-1 gag variant peptides as indicated or (C) with different doses of index peptide. Apoptosis of both CTL and target was determined after an 12-hour incubation as above. (D) Influenza matrix specific A2-restricted CTL (Flu-CTL) were cocultured with T2 cells stably transfected with the β2M/FluMP58-66 matrix fusion construct at different E:T ratios. CTL death was assessed at by Annexin-V staining and death of targets by an 8-hour ⁵¹Cr release assay.

FIG. 2. Induction of Apoptosis by Multimeric MHC Class I Complexes.

(A) Flu-CTL were cultured for 12 hrs with beads (5 beads per cell) coated with FluMP58-66-A2wt or control irrelevant (Irrel-A2wt) tetramers and death of CTL was assessed by Annexin-V staining. (B) Dose-dependent (from 0 to 10 beads/cell) induction of the Flu-CTL apoptosis by the flu-A2wt tetramer beads assessed by Annexin-V staining. (C) Density-dependent induction of CTL apoptosis. Mixed multimers were formed by loading streptavidin coated beads with different molar ratios of two A2 monomers (flu-A2 wild type and Irrel-A2 wild type). These “mixed multimers” were then assessed for the induction of CTL apoptosis as above.

FIG. 3. Mediators of CTL Death

Flu-CTL were cultured for 12 hrs with Flu-A2wt multimeric beads in the presence of either: IL-2 (20 U/ml), anti-CD28 (1 μg/ml) or autologous/allogeneic B cells. The apoptosis of CTL was then determined by JAM assay (³H-thymidine incorporation) as described in the experimental procedures. B) Inhibition of CTL apoptosis (assessed by Annexin-V or TUNEL) using caspase inhibitors or soluble Ig-Fc chimeras of the indicated death receptors.

FIG. 4. The Role of CD8 in CTL Function and Apoptosis

(A) Effect of anti-CD8 antibody on death of CTL and targets. Flu-CTL were cocultured with untransfected JY cells (A2 positive B cell line) or JY transfected with the Flu-β2M fusion construct (JY/Flu). Cells were incubated in the presence or absence of a blocking anti-CD8 mAb (MF8 1/1000 dilution of ascites) at different CTL:target ratios. After 8 hours culture, apoptosis of CTL was determined by Annexin-V staining of T cells gated using anti-CD3. In parallel the death of target cells was determined, at the same time point, by a ⁵¹Cr-release.

B) and C). Effect of MHC class I α3 mutants on the death of CTL and targets. Flu-CTL were cocultured with A2 wild type (A2 wt) or A2 mutant (A2mt) expressing 0.221 cells pulsed with or without FluMP58-66 peptide (subsequently washed) at different CTL:target ratios. Similarly the B4402 alloreactive CTL (LC13) were cocultured with either B4402wt or B4402mt expressing cells and the death of CTL and targets were determined as described above. D). Residual cytotoxicity induced by the MHC class I α3 mutants is mediated by FasL. Soluble Fas-Fc fusion proteins (20 μg/ml) or anti-FasL neutralising mAb (5 μg/ml) were added to the culture as described in B and C.

FIG. 5. Induction of Apoptosis of CTL by Multimeric MHC Class I α3 Mutant Complexes.

A). Tetramer staining and death induction by mutant Flu-A2 multimeric beads which do not bind CD8. Flu-CTL were stained with control, wild type or α3 mutant tetramers (5 μg/ml, 30 min at 37° C. followed by staining with anti-CD8 tricolour) (left panel). Apoptosis of CTL was assessed by Annexin-V staining following a 12-hour incubation with control, wild type or mutant multimeric beads (5 beads/cell) (right panel).

B) Dose-dependent binding to tetramers to CTL.

C) Induction of apoptosis of CTL by the mutant multimers. Experiments were performed as described in FIGS. 2A &B.

FIG. 6. Signalling and Apoptosis Induced by Wild Type and Mutant MHC Complexes.

A) Phosphorylation of the TCR zeta chain induced by wild type or mutant MHC complexes. Flu-CTL were cultured with beads coated with A2 monomers for up to 30 mins. Zeta chain phosphorylation was detected by Western blotting with an anti-phosphotyrosine mAb.

B) Comparison of CTL death with zeta chain phosphorylation. Beads were coated with the indicated molar ratios of wild type vs irrelevant (lanes 1-5: 4/0, 3/1, 2/2, 1/3, 0/4, (+) respectively) or vs mutant (lanes 6-9: 3/1, 2/2, 1/3, 0/4, (+) respectively) A2 complexes. CTL were incubated with the beads for 5 min and zeta phosphorylation measured as above, or incubated for 12 hours and apoptosis measured by annexin-V staining. As a control for protein loading western blots were stripped and reprobed with an antibody to (A) β-actin or total ZAP-70 (B).

FIG. 7. Study of Polyclonal CMV-Specific CTL Response.

PBMC from a normal CMV positive HLA-A2⁺ individual were incubated for 6 hrs with B cells pulsed with irrelevant or CMV matrix peptide (NLVPMVATV). CMV-A2 specific CTL in this mix of PBMC were identified using a CMV-A2 tetramer (see methods). Apoptosis of the tetramer positive cells was determined by counterstaining with Annexin-V-FITC and anti-CD3-tri-colour.

B) The effect of blocking CD8 on CMV specific CTL death. Cells were cultured as above at varying E:T ratios (PBMC:B cells) in the presence or absence of blocking anti-CD8 mAb. Apoptosis of CMV-A2 cells was assessed by Annexin-V staining of the CD3/tetramer gated cells shown in (A).

FIG. 8. Illustration of the Different Pathways Following Disruption of CD8 Binding.

A) shows the normal events following interaction between a CTL and a MHC Class I molecule displaying peptide antigen.

B) shows the events following interaction between a CTL and a MHC class I molecule displaying peptide antigen where the CD8 binding is prevented.

FIG. 9. Specific Killing of CTL Using Mutant MHC I Molecules Deficient in CD8 Co-Receptor Binding.

This figure shows the effects of wild type and mutant MHC class I molecules when expressed at the cell surface.

FIG. 10. shows the results of a ⁵¹chromim and CTL assay performed with an Anti-A2-Flu specific cytotoxic T cell clone (methodology described in Xu et al. Immunity Vol 14 pages 591-602, 2001).

### DETAILED DESCRIPTION

The MHC codes for three families of glycoproteins known as Class I, Class II and Class III MHC molecules. The Class I and Class II MHC molecules are expressed mainly as membrane glycoproteins at the cell surface. One of the important features of MHC molecules in their polymorphism. That is, within each class of molecules and even at one locus, a large number of variants (polymorphic forms or alleles) exists in the population as a whole. Thus, for a population of people there will be many genes for each type of product, each coding for a separate MHC allele or variant. However, each individual only has a very small set of different MHC genes and expresses a movement of two alleles for each locus.

The products of the Class I MHC genes (e.g. Human Leukocyte Antigen (HLA)—A, B, and C loci in humans) are membrane glycoproteins. Each Class I molecule is a heterodimer composed of an a or heavy chain polypeptide and a β2 microglobulin (β2M), which is noncovalently associated with the α chain.

The Class I α chain is polymorphic and encoded within the MHC, whereas polymorphism of β2M is limited (only one allele has been identified in human and seven in mouse). The β2M gene is not encoded by the MHC.

The α chain is a transmembrane polypeptide chain that can be divided into five distinct structural regions or domains. Three of these domains, α1, α2 and α3 are exposed on the outside of the cell and are known as the extracellular domains. The α3 domain is found closest to the plasma membrane. The β2M is associated with the extracellular portion of the α chain and sits on the membrane next to the α3 domain.

As well as providing information about how MHC molecules might present antigens to T cells, structural analysis has also allowed the parts of the molecule that stimulate antibody production to be identified. Antibodies have been used to determine the HLA alleles expressed by different individuals. The technique is known as HLA typing.

For certain diseases, an increased frequency of particular HLA alleles has been noted in affected individuals. Many autoimmune diseases show an increased frequency with particular alleles.

As mentioned above, the present inventors have devised a method by which unwanted immune responses can be selectively reduced or prevented. This method is based on the discovery that peptide/MHC complexes created with mutant heavy chain lacking CD8 interaction delete specific CTL populations without the concomitant T cell activation.

The method of the invention could be used both when the peptides are known, but perhaps more usefully, it could be extended to instances where the peptides are not known but the responsible MHC molecules are or at least can be determined without undue difficulty. An example of this is organ transplantation where a significant amount of rejection is caused by allo-specific responses to the donor MHC. If these MHC molecules are mutated in the CD8 binding domain (if class I) or the CD4 binding domain (if class II) then it will be possible to control the immune responses without knowledge of the peptides. Such peptide-independent yet specific therapy would also be possible in autoimmune disease where the restricting MHC molecules were known or could be determined.

Experimental Procedures

CTL Clones and Target Cells

Three HLA-A2-restricted and one HLA-B8-restricted CD8⁺ CTL clones were used in this study; the 868 clone specific for HIV-1 gag epitope (SLYNTVATL), two influenza-specific clones (9C and Nikila) for matrix protein 58-66 (GILGFVFTL), and the LC13 clone specific for the EBV epitope FLR and alloreactive to HLA-B4402, have been described previously [Dunbar, 1998; Tan, 1999; Burrows et al]. Targets used in this study were EBV-transformed B cell lines (BCL), the T2 cell line (A2⁺ Tap⁻) and T2 or BCL's stably transfected with human β2 microglobulin fused to the Flu matrix peptide 58-66 (FluMp⁵⁸⁻⁶⁶) using a retroviral vector as described previously (Ulta et al). The 0.221 cell lines (MHC Class I negative) stably expressing HLA-A2 wild type (A2wt), B4402 wild type (B4402wt), or their mutants (A2mt or B4402mt) were made by transfection with full length cDNA clones fused to GFP in pEGFP-N1 (Clontech, UK). Mutants of A2 and B4402 were generated by substitution of amino acids D and T at position 251 and 252 with K and A (251D/252T) in the α3 domain on the basis of structure of CD8 and HLA-2 interaction [Gao, 1997]. The vectors were transfected into 0.221 cells by electroporation and subsequently cultured in the presence of selection antibiotic neomycin (G418, Gibco). The GFP positive cells were then sorted and stained with anti-class I mAb (W6/32) to confirming equal cell surface expression.

Peptides and Tetramers

The HLA-A2 restricted natural variant peptides from HIV-1 gag p17-8, 3F (SLFNTVATL), 3S (SLSNTVATL), 3H (SLHNTVATL) and 3F5A (SLFNAVATL) [Sewell, 1997] were used to pulse autologous B cells at 5 μM or as indicated for 2 hours. BCL's were then washed 3 times and resuspended for use as targets. Production of MHC-peptide tetrameric complexes has been described [Altman, 1996]. Briefly, purified HLA-A2 heavy chain and β2M/peptide fusion proteins were produced in E. coli BL21 DE3 pLysS using pET expression vectors. The BirA biotinylation site was added to the C-terminus of A2. The A2 heavy chain and β2M/peptide fusion were refolded using standard buffers. The 45 kD refolded product was isolated by FPLC and biotinylated with BirA. For cell surface staining tetramers were prepared by mixing monomers with streptavidin-phycoerythrin (PE) conjugate (Sigma *E-4011) at a molar ratio of 4:1. For cell culture, we used magnetic multimeric beads prepared by mixing an excess of monomers with beads coated avidin (7×10⁵ molecules/bead, Dynabeads M-280). To reduce the proportion of A2 wild type MHC complexes we mixed beads with optimal amounts of FluMP⁵⁸⁻⁶⁶ A2 monomers and irrelevant (melanoma) A2 monomers at molar ratio of 3:1, 2:2, 1:3 respectively. All beads were washed 3 times with culture medium to remove Sodium Azide before the assays.

Tetramer Staining

For tetramer staining cells were incubated at 37° C. for 15-30 min to allow TCR-mediated binding and rapid internalisation of the tetramer [Whelan, 1999]. After washing cells were counterstained with additional antibodies such as anti-CD8 tricolour (*MHCD0806, Caltag) or anit-CD3 tricolour (*MHCD0306, Caltag). As this step is done following binding and internalisation of tetramer the possibility that anti-CD8 or anti-CD3 may block tetramer binding is avoided.

For the analysis of the CMV-A2 specific polyclonal CTL, freshly-purified PBMC were pre-stained with the CMV-A2 wild type tetramers for 15 min at 37° C. This short “pre-labelling” of antigen-specific CTL prior to activation has been used to overcome the problem of TCR down-regulation after antigen stimulation [Appay, 2000]. The short incubation with tetramer did not induce CTL apoptosis above background levels (data not shown). After washing, cells were incubated for 6 hours at 37° C. in the 96-well round bottom plates with targets. The B cell targets were pulsed and washed (to remove free peptide) with index CMV matrix peptide (NLVPMVATV) or irrelevant peptide at different PBMC:Target ratios (200:1, 100:1, 50:1). Different concentrations (1/1000, 1/500 and none) of blocking anti-CD8 antibody (MF8 ascites) were added to the cultures. At the end of the assay, cells were washed and stained with anti-CD3 tricolour and Annexin-V FITC (*1828-681, Boehringer Mannheim).

Apoptosis Assays

CTLs were incubated with target cells at different effector: target (E:T) ratios as indicated, or with the multimeric beads (5 beads/cell or as indicated) in 96-well flat bottom plates in a final volume of 200 μl for the time indicated. The cells were then harvested for apoptosis assays. For inhibition of apoptosis, reagents were added at the beginning of the assay at a final concentration of rIL-2 (20 U/ml), anti-FasL mAb (5 μg/ml, kindly provided by Dr Nagata), anti-TNF neutralising mAb (50 μg/ml), Ig-fusion proteins (20 μg/ml), caspase-inhibitors (z-VAD and the control z-FA-FK, 10 μM; Enzyme Systems), or a blocking anti-CD8 mAb (MF8), Three methods for detecting apoptotic cells were used: 1) Annexin-V staining or TUNEL assay: CTL were labelled with anti-CD8 (Tricolour, Caltag) and BCL targets with anti-CD19 (PE, Dako) and then stained with Annexin-V-FITC or TUNEL (FITC) according to the manufacturers protocols. 2) JAM assay: briefly the CTLs were cultured with ³H-TdR (1 μCi/ml) for 12-24 hours, washed 3 times, and then cultured with the multimeric beads for another 12-hours. DNA fragmentation was determined by ³H-Thymidine release [Matzinger, 1991]. 3) Finally death of target cells was also assessed by a standard ⁵¹Cr release assay.

Although the apoptosis of CTL and the target can be monitored simultaniously by 3-colour flow cytometry, we found that Annexin-V staining was more reliable and sensitive for analysis of CTL apoptosis because CTL were not labelled well with ⁵¹Cr (they often show high spontanous release). On the other hand, the ⁵¹Cr release assay was used to monitor apoptosis of target cells because most BCL including 0.221 gave a high back ground staining with Annexin-V (data not shown).

TCR Signalling

2×10⁶ FluMP58-66-specific CTLs were incubated with multimeric beads (5 beads/cell) for the time indicated at 37° C. Cells were then lysed as previously described [Purbhoo, 1998], and the lysate cleared by centrifugation and resolved by SDS-PAGE. Following transfer the Western blot was probed with the anti-phosphotyrosine mAb (4G10 Upstate Biotechnology) followed by a secondary antibody conjugated with HRP and revealed using enhanced chemiluminescence (ECL). Equal loading of lanes was demonstrated by re-probing the blot with a monoclonal antibody to total cellular ZAP70 (Santa Cruz) or β-actin (Sigma).

Results

Correlation of Death of the CTL and the Targets

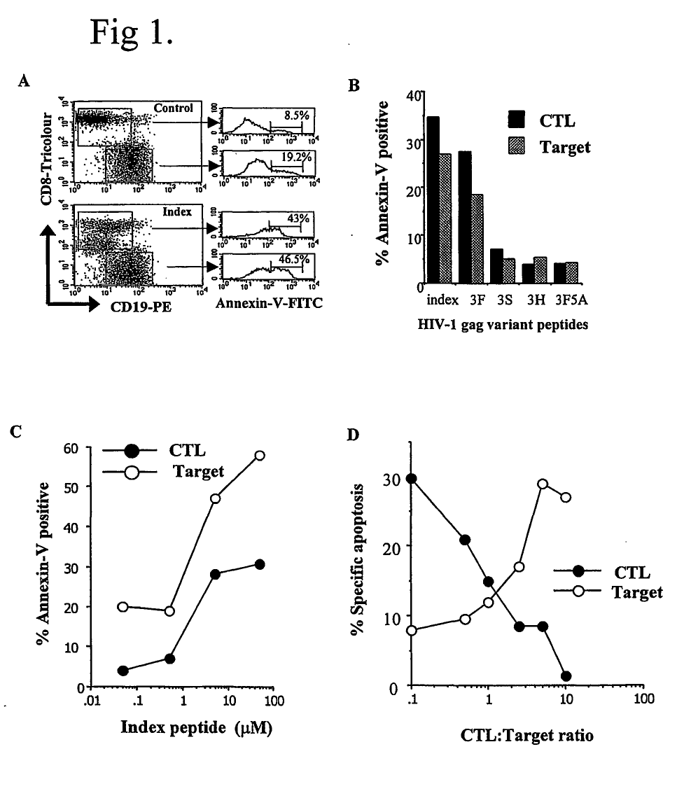

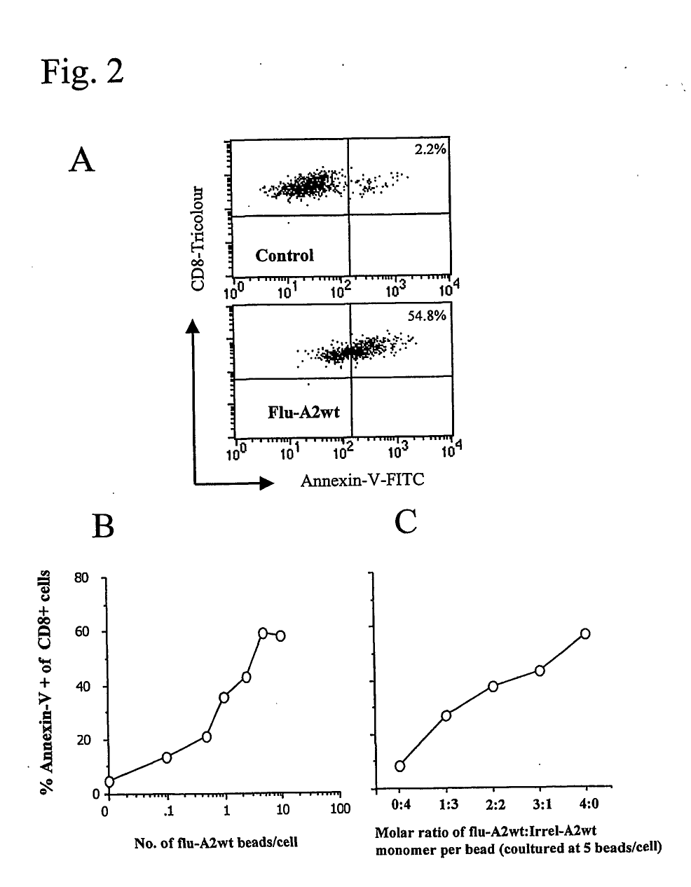

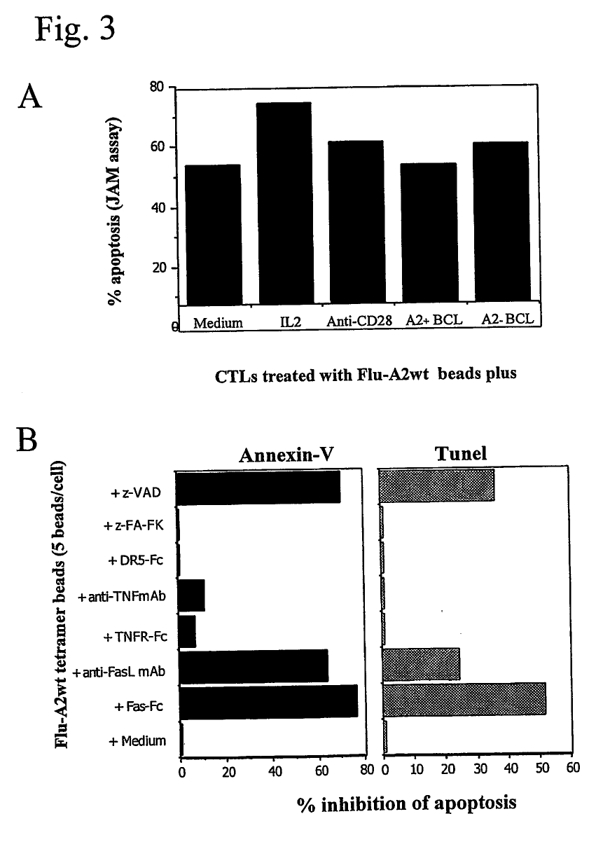

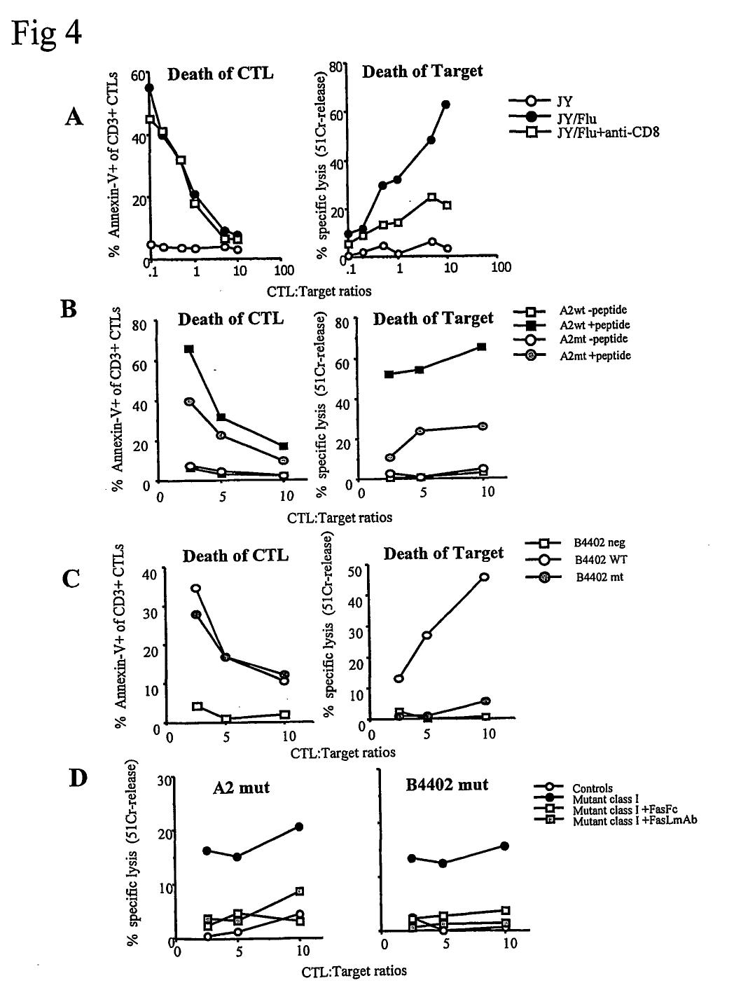

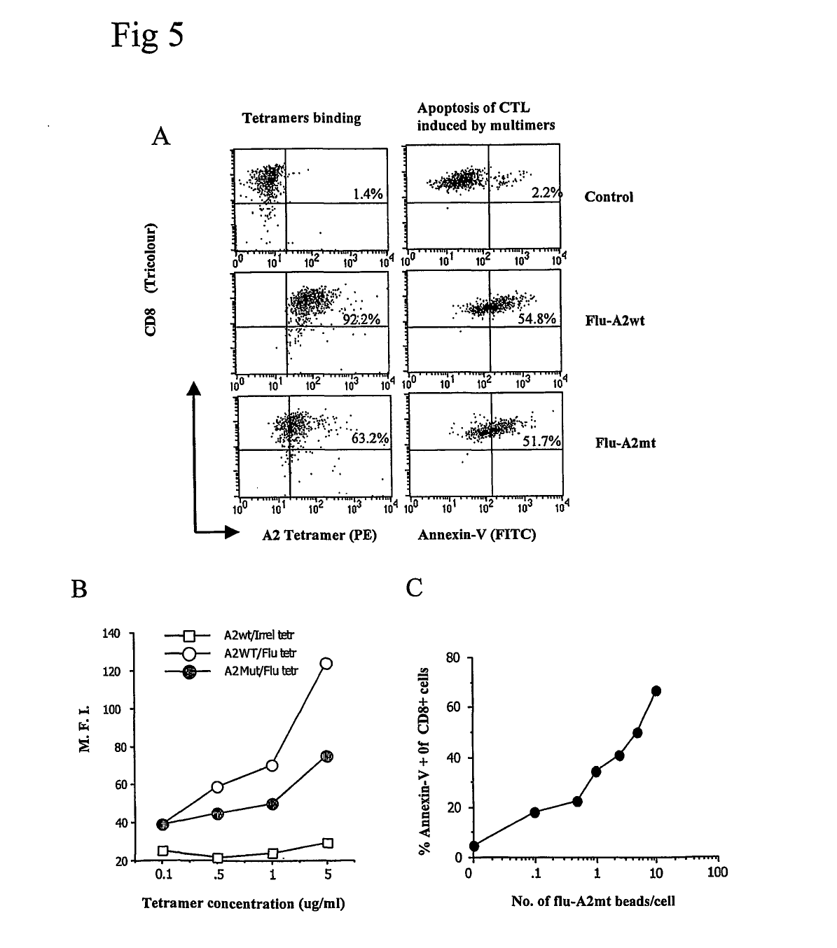

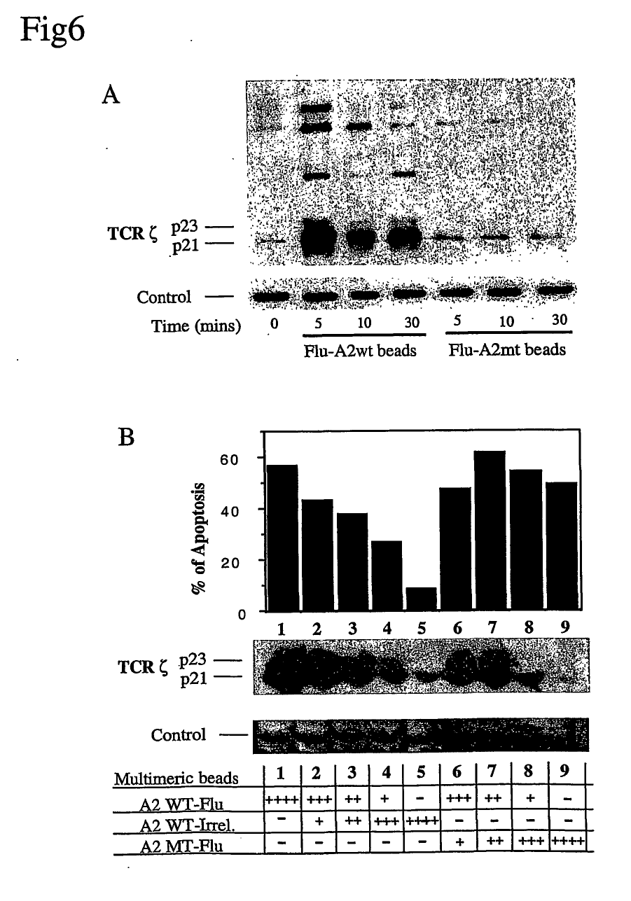

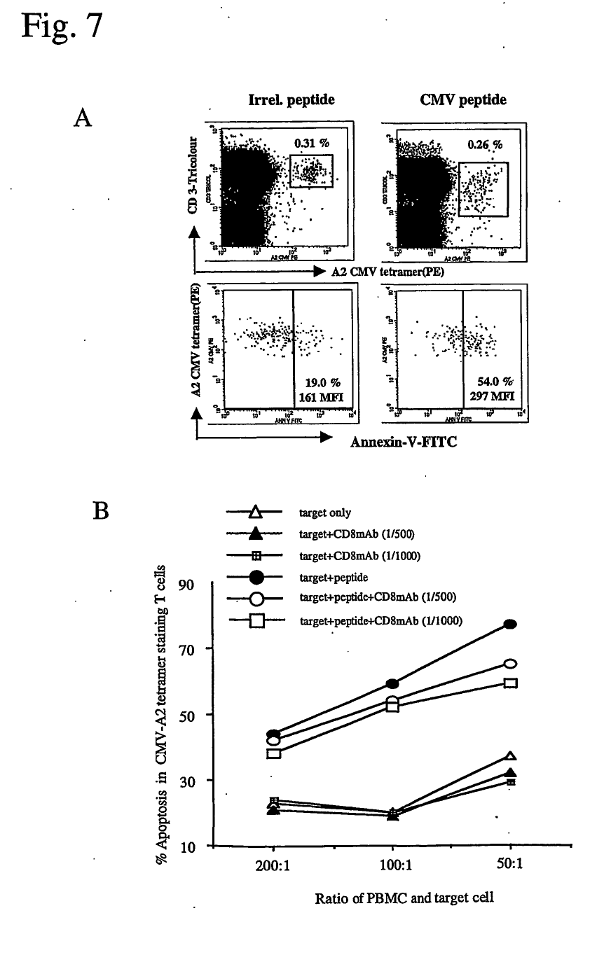

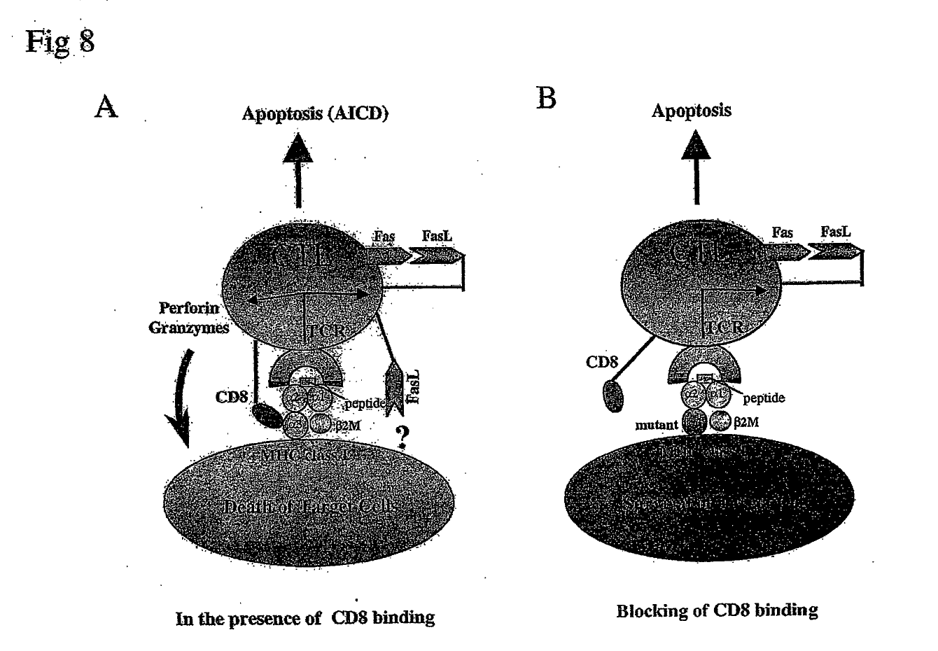

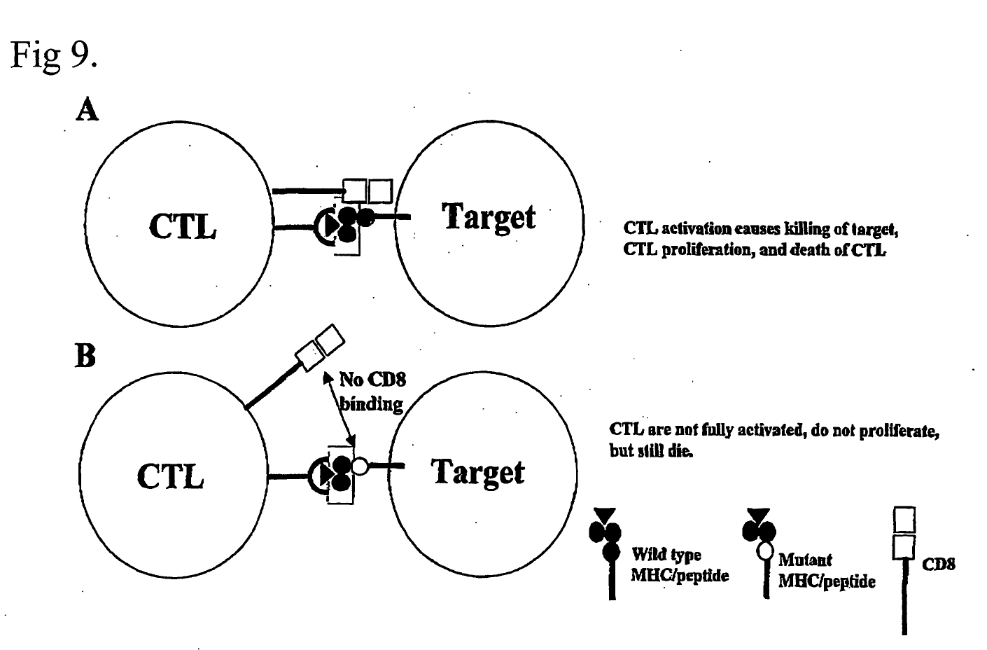

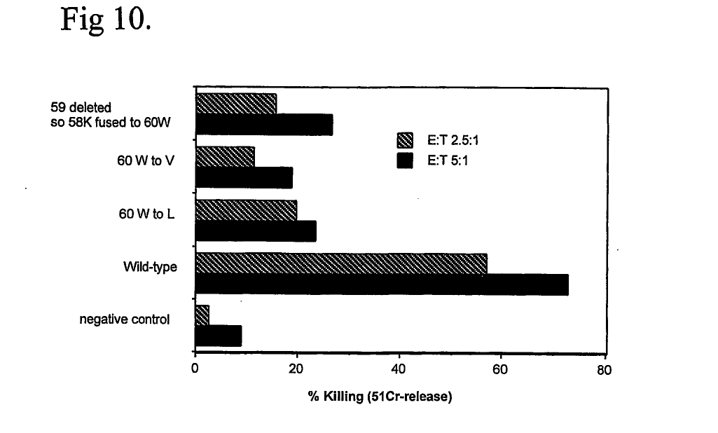

To investigate the balance between the killing activity and the death of CTL the inventors performed CTL assays in which both the death of the CTL and death of their targets were measured. An HIV-1 gag-specific, HLA-A2-restricted T cell clone was incubated with A2⁺ B cell targets pulsed (and subsequently washed) with index (SLYNTVATL) peptide at an E:T ratio of 2:1. After 12 hrs incubation the apoptosis of CTLs and targets was measured by tri-staining with anti-CD8-Tricolour, anti-CD19-PE, and Annexin-V-FITC. Using the gag index peptide the inventors saw up to 27% specific killing of targets and 35% death of the CTLs. A representative experiment is shown in FIG. 1a, interestingly following stimulation with index peptide the CTL distribute between CD8 high and CD8 low populations and most of the apoptotic cells (80%) reside in this latter population. Next the inventors used a range of naturally observed variant gag epitopes. The variants have comparable binding affinities for MHC A2 but differ in their abilities to activate CTL [Sewell, 1997]. There was a good correlation between target cell and CTL death for these variants with both the index and 3F sensitising both target and CTL to death whilst variants 3S and 3H had no effect on either (FIG. 1b). Interestingly, 3F5A, an antagonist for the index [Sewell, 1997], had no effect on CTL death. Titration of the index peptide showed similar effects on CTL and target cell death (FIG. 1c).

One explanation for the above results was that CTL were killing each other in a peptide-specific fashion by binding residual peptide left in solution (even though targets were extensively washed and supernatant from a CTL assay did not cause death when transferred onto fresh CTL, data not shown). To exclude formally this possibility the inventors developed a system free of added peptide. A flu matrix peptide was covalently linked to β2M by cloning the corresponding DNA sequences onto its N-terminus. T2, an A2 cell line deficient in the TAP genes and lack of cell surface expression of MHC class I, was stably transfected with the β2M fusion. The transfected cells were then used as targets in a flu-specific CTL assay. The results again demonstrated considerable death of CTL confirming their original observations with the Gag clone. In addition the inventors performed the assay at E:T ratios from 0.1-10:1. Decreasing the effector:target ratio dramatically increased the apoptosis of the CTLs (presumably by maximising their exposure to antigen), whereas increasing the ratio leads to better CTL survival and a concomitant increase in the efficiency of target cell killing (FIG. 1d). Interestingly the two lines cross at an E/T ratio close to 1:1, suggesting that in these assays, on average one CTL only killed a single target.

CTL Death can be Induced by Peptide/MHC Multimeric Complexes.

Peptide-MHC tetramers enhance the avidity of TCR binding and have been used extensively as staining reagents to analyse specific T cell populations and also to study T cell activation [Boniface, 1998]. The inventors took advantage of this technology to produce a target cell free system to assess the contribution of adhesion/accessory molecules to the death of CTL. They constructed multimers using the A2 heavy chain complexed with the β2M/FluMP⁵⁸⁻⁶⁶ fusion protein as described above. These multimers efficiently triggered death of Flu-specific (MP58-66) CTL (FIG. 2a). Death of CTL induced by magnetic beads saturated with the multimers (FIG. 2b) showed a similar dose response curve to that obtained using the T2 targets described above (FIG. 1d). It has previously been shown that reducing the stochiometry of the MHC:streptavidin reduced T cell activation/signalling, [Savage, 1999]. The inventors reduced the loading of beads with agonist monomer to 75, 50, 25 and 0% of maximum by varying the ratio of two A2 complexes containing either index (Flu-MP58-66) peptide or an irrelevant peptide. Reducing the loading of FluMP⁵⁸⁻⁶⁶ MHC from 100% to 25% reduced CTL death (FIG. 2c).

A number of cytokines and costimulatory molecules have been suggested to play a role in the modulation of activation induced cell death. To look for possible modulators, the inventors took two approaches: firstly, CTL were incubated with multimer beads in the presence of either IL-2 or anti-CD28. Anti-CD28 was without effect whilst IL-2 in some cases led to a small increase in CTL death. In addition, to provide a source of co-stimulatory interactions, they added irrelevant APC's to the culture. Neither autologous nor allogeneic APC's reduced the death of CTL (FIG. 3a).

To examine the mechanism of CTL death, the inventors included caspase inhibitors in the CTL assay. z-VAD blocked the tetramer-induced apoptosis of CTL implying a caspase dependent apoptotic pathway (FIG. 3b). Next they incubated cells with soluble IgFc chimeras to block the death induced by FasL, TNF, or TRAIL. Consistent with studies on CD4⁺ T cells, Fas-FasL interaction plays a central role in the death of these human CTL clones because Fas-Fc or anti-FasL mAb inhibited up to 80% of CTL death.

CD8 is not Required to Signal CTL Death.

In a further search for molecules regulating the balance between target and CTL death, the inventors examined the role of the co-receptor CD8. Most human CTLs kill their targets in a CD8 dependent fashion (i.e. blocking CD8 blocks/reduces CTL killing). Anti-CD8 was added to a CTL assay using, as targets, the A2 positive B cell line JY transfected with the FluMP⁵⁸⁻⁶⁶/β2M fusion construct. These assays confirmed that blocking CD8/MHC interaction, over a range of E:T ratios, reduced the killing of targets by up to 80%. Interestingly, over the same time period, there was little effect on the death of CTL (FIG. 4a).

Anti-CD8 antibodies have been shown to either block or augment TCR-MHC/peptide interactions depending on binding to different epitopes on CD8 [Daniels, 2000]. To further characterise this effect, mutants of MHC class I (A2 and B4402) were constructed. Based on the CD8-HLA-A2 structure [Gao, 1997] two amino acid substitutions (aa 227/228, from DT to KA) were made in the α3 domain which were predicted to abolish the interaction. As expected this mutation abrogates binding to CD8 measured using Biacore analysis (Purbhoo et al unpublished data). Wild type and mutant A2 molecules were stably expressed in 0.221 cells, which lack MHC class I, and comparable levels of expression were verified by FACS analysis (data not shown). When pulsed with Flu index peptide the killing of targets expressing mutant A2 was reduced to a similar degree as blocking with anti-CD8 mAb. Remarkably, in spite of the lack of interaction with CD8, the A2 mutant expressing cells were still competent to induce death of Flu-specific CTL (FIG. 4b). Next the inventors tested whether this system could apply to alloreative CTL in which the endogenous antigenic peptide remains unknown. The inventors studied the well-characterized EBV-specific HLA B8-restricted CTL clone (LC13) which cross-recognises HLA B4402 [Burrows, 1999]. Cells expressing B4402 wild type or the B4402 mutant lacking CD8 binding were equally competent at causing AICD of the alloreactive CTL, however killing of targets expressing the mutant B4402 was greatly reduced.

0.221 cells express Fas and are very sensitive to killing by FasL, so we reasoned that stimulation of T cells with mutant MHC lacking the ability to interact with CD8 may abolish granule release, but that some residual killing activity may be mediated by FasL. CTL assays with targets expressing mutant A2 or B4402 were repeated in the presence of soluble Fas or neutralising anti-FasL mAb. These two blocking reagents inhibited the residual CTL activity to background levels indicating that only FasL killing is activated in CTL stimulated without CD8 co-receptor engagement (FIG. 4d).

To further confirm the results of cells expressing MHC class I mutant, the inventors also tested the activity of the β3 domain mutation of A2 in the target free assay system by constructing MHC complexes containing mutant A2 complexed to the βM-Flu fusion protein (similar to the A2wt-β2M/FluMP⁵⁸⁻⁶⁶ tetramer as described above). The mutant tetramer was still able to stain the FluMP⁵⁸⁻⁶⁶CTL clone, although to a reduced intensity (FIG. 5b). This reduction likely reflected a reduced binding avidity as it was similar to that seen with a blocking anti-CD8 mAb if applied before the tetramer staining (data not shown). In spite of the weaker binding, beads loaded with the A2 mutant complexes were still fully competent to induce death of CTL (FIG. 5a), and this showed a similar dose-response to beads loaded with wild-type complexes (FIG. 5c).

Apoptosis of CTL Induced by the MHC Class I Mutant is Independent of TCR Zeta-Chain Phosphorylation.

The induction of AICD in the absence of CD8 interaction was surprising as blocking CD8 has been shown to inhibit TCR signalling as determined by phosphorylation of TCR zeta or Zap70 [Kersh, 1998]. CTL were exposed to beads coated with wild type and mutant A2-Flu complexes and tyrosine phosphorylation was measured by western blotting. Compared to control, mutant A2 triggered little Zeta chain phosphorylation whilst wild type beads triggered considerable phosphorylation over the indicated time course (FIG. 6A). In a further series of experiments the inventors loaded beads with a mixture of wild type/irrelevant or wild type/mutant A2 complexes and measured zeta phosphorylation by western blot and also death of the CTL by Annexin V staining. When the wild type complexes are sequentially replaced by irrelevant complexes there is a good correlation between CTL apoptosis and the level of T cell activation as evidenced by Zeta chain phosphorylation (FIG. 6B lanes 1-5). However as the wild type complexes are replaced by the α3 domain mutant A2 complexes the level of apoptosis remains constant whilst tyrosine phosphorylation is much reduced (FIG. 6B lanes 1 and 6-9). Thus, zeta phosphorylation and CTL death appear to have been dissociated by blocking interaction with CD8. In the absence of CD8 binding. The reduced signalling/activation induced by the mutant multimers was also evidenced by a reduced expression of the early activation marker CD69 when compared with CTL stimulated with wild type multimers (data not shown).

Death of CTL in Response to Antigen is Also Found to be CD8-Independent in Polyclonal CTL Responses.

The experiments detailed above examined apoptosis of CTL clones maintained in long term culture. To confirm that these results were applicable to polyclonal CTL responses in vivo, the inventors examined an HLA-A2 restricted anti-CMV response from a healthy individual. Fresh isolated PBMC were co-cultured with peptide pulsed targets in the presence or absence of blocking anti-CD8 mAb in experiments analogous to those depicted in FIG. 4a.

The A2 CMV-specific CTL population was assessed by staining with a CMV-A2 tetramer. As expected, gating on this population revealed that stimulation of cells with CMV peptide pulsed targets (subsequently washed several times) for 6 hours in the absence of CD8 led to significant CTL death when compared with targets pulsed with irrelevant peptide (FIG. 7a). Across a range of E:T ratios, in the presence of the blocking anti-CD8 mAb, at the same or double the concentration used to block the CTL clones, CTL death was not blocked confirming their results using the FluMP⁵⁸⁻⁶⁶-A2 specific CTL clones (FIG. 7b).

Modification of β2M of MHC Class I

The β2M component of MHC class I molecules is capable of folding correctly in the absence of the a heavy chain. Therefore, the inventors have determined that the β2M component may be modified in accordance with the present invention to generally suppress or to selectively suppress the immune system in a patient. The β2M is preferably modified at residues 57 and 61, more preferably 58-60, particularly lysine 58 as this makes contact with an arginine in CD8 in both human and mouse crystals. It has been shown that mutation of arginine which contacts this in murine CD8 abolishes binding.

Administration of a modified 2M would allow general immunosuppression. However, if specific/selective immunosuppression was required, one could administer a peptide linked to the modified β2M. The β2M peptide linkage allows the delivery of stable complexes which are completely specific.

Mutants of Beta 2 microglobulin were created by PCR amplification (primer sequences shown below) with a reverse primer that contained changes to amino acids between 57 and 61 which are underlined in the following sequence.

IQRTPKIQVYSRHPAENGKSNFLNCYVSGFHPSDIEVDLLKNGERIEKVE
HSDLSFSKDWSFYLLYYTEFTPTEKDEYACRVNHVTLSQPKIVKWDRDM

Mutants were cloned into the expression vector pCDNA3 (Invitrogen). Two constructs for each mutant were made: Firstly the whole coding sequence (with mutant) of B2M was used (full length wild type sequence of B2M shown below):

MSRSVALAVLALLSLSGLEAIQRTPKIQVYSRHPAENGKSNFLNCYVSGF
HPSDIEVDLLKNGERIEKVEHSDLSFSKDWSFYLLYYTEFTPTEKDEYAC
RVNHVTLSQPKIVKWDRDM

Additionally, fusions between the mutant B2M sequences were made into a vector in which sequences encoding the Influenza Nucleoprotein peptide sequence and a synthetic linker was inserted between the B2M leader and mature protein sequence (Xu et al. Immunity Vol 14 pages 591-602 2001). The sequence of this without additional mutation is shown below and the inserted sequences highlighted:

MSRSVALAVLALLSLSGLEGGILGFVFTLGGGSGGGGSGGSGGSGGIQRT
PKIQVYSRHPAENGKSNFLNCYVSGFHPSDIEVDLLKNGERIEKVEHSDL
SFSKDWSFYLLYYTEFTPTEKDEYACRVNHVTLSQPKIVKWDRDM

To test for expression the mutant B2M clones (without the added Flu peptides) were transiently transfected into mouse L cells using Lipofectin (invitrogen). 24 hours later expression of human B2M was assessed using a monoclonal antibody specific for human B2M (BBM1). The results are shown in Table I below.

For mutants in which cell surface expression was seen, a second series of experiments were performed. Human 293T cells (which express HLA A2.1) were transfected by calcium phosphate with the mutant B2M constructs which also have an N-terminally linked Flu peptide described above. 24 hrs after transfection the cells were labelled with ⁵¹Chromim and a CTL assay was performed with an Anti-A2-Flu specific cytotoxic T cell clone (methodology described in Xu et al. Immunity Vol 14 pages 591-602 2001). The results of the killing assay are presented in the table and specific examples where inhibition was seen are shown in the histogram (FIG. 10.)

It should be noted that the B2M sequences that are not expressed at the cell surface may still be able to block killing. To test this mutants can be expressed in E. Coli, refolded from inclusion bodies and then used to pulse cells which can then be used as targets in a chromium release CTL assay.

To test for mutants that block CTL killing but which are also able to kill the CTL the death of CTL can be tested by standard methods such as previously described in Xu et al. Immunity Vol 14 pages 591-602 2001

TABLE I

| | | inhibition |
|---|---|---|
| | expression | of killing |
| Beta 2 microglobulin Mutant | in L cells | of target c |
| 58 K-N | | |
| ggtgaattcagtgtagtacaagagata | Yes | |
| gaaagaccagtcGTTgctgaaagacaa | | |
| gtctg | | |
| 58 K-C | | |
| ggtgaattcagtgtagtacaagagata | Yes | No |
| gaaagaccagtcGCAgctgaaagacaa | | |
| gtctg | | |
| 58 K-Q | | |
| ggtgaattcagtgtagtacaagagata | | No |
| gaaagaccagtcCTGgctgaaagacaa | | |
| gtctg | | |
| 58 K-E | | |
| ggtgaattcagtgtagtacaagagata | Yes | No |
| gaaagaccagtcCTCgctgaaagacaa | | |
| gtctg | | |
| 58 K-G | | |
| ggtgaattcagtgtagtacaagagata | Yes | No |
| gaaagaccagtcGCCgctgaaagacaa | | |
| gtctg | | |
| 58 K-H | | |
| ggtgaattcagtgtagtacaagagata | | |
| gaaagaccagtcGTGgctgaaagacaa | | |
| gtctg | | |
| 58 K-I | | |
| ggtgaattcagtgtagtacaagagata | | |
| gaaagaccagtcGATgctgaaagacaa | | |
| gtctg | | |
| 58 K-L | | |
| ggtgaattcagtgtagtacaagagata | Yes | |
| gaaagaccagtcCAGgctgaaagacaa | | |
| gtctg | | |
| 58 K-M | | |
| ggtgaattcagtgtagtacaagagata | Yes | No |
| gaaagaccagtcCATgctgaaagacaa | | |
| gtctg | | |
| 58 K-F | | |
| ggtgaattcagtgtagtacaagagata | Yes | No |
| gaaagaccagtcGAAgctgaaagacaa | | |
| gtctg | | |
| 58 K-P | | |
| ggtgaattcagtgtagtacaagagata | Yes | No |
| gaaagaccagtcGGGgctgaaagacaa | | |
| gtctg | | |
| 58 K-S | | |
| ggtgaattcagtgtagtacaagagata | Yes | |
| gaaagaccagtcGCTgctgaaagacaa | | |
| gtctg | | |
| 58 K-T | | |
| ggtgaattcagtgtagtacaagagata | Yes | No |
| gaaagaccagtcGGTgctgaaagacaa | | |
| gtctg | | |
| 58 K-W | | |
| Ggtgaattcagtgtagtacaagagata | Yes | No |
| gaaagaccagtcCCAgctgaaagacaa | | |
| gtctg | | |
| 58 K-Y | | |
| ggtgaattcagtgtagtacaagagata | Yes | No |
| gaaagaccagtcGTAgctgaaagacaa | | |
| gtctg | | |
| 58 K-V | | |
| Ggtgaattcagtgtagtacaagagata | Yes | No |
| gaaagaccagtcCACgctgaaagacaa | | |
| gtctg | | |
| 60 W-R | | |
| Ggtgaattcagtgtagtacaagagata | | |
| gaaagaCCGgtccttgctgaaagacaa | | |
| gtctg | | |
| 58 K-S 60 W-R | | |
| ggtgaattcagtgtagtacaagagata | No | |
| gaaagaCCGgtcGCTgctgaaagacaa | | |
| gtctg | | |
| 60 W-N | | |
| ggtgaattcagtgtagtacaagagata | | |
| gaaagaGTTgtccttgctgaaagacaa | | |
| gtctg | | |
| 60 W-C | | |
| ggtgaattcagtgtagtacaagagata | No | |
| gaaagaGCAgtccttgctgaaagacaa | | |
| gtctg | | |
| 60 W-Q | | |
| ggtgaattcagtgtagtacaagagata | No | |
| gaaagaCTGgtccttgctgaaagacaa | | |
| gtctg | | |
| 60 W-E | | |
| ggtgaattcagtgtagtacaagagata | No | |
| gaaagaCTCgtccttgctgaaagacaa | | |
| gtctg | | |
| 60 W-G | | |
| ggtgaattcagtgtagtacaagagata | No | |
| gaaagaGCCgtccttgctgaaagacaa | | |
| gtctg | | |
| 60 W-H | | |
| ggtgaattcagtgtagtacaagagata | Yes | No |
| gaaagaGTGgtccttgctgaaagacaa | | |
| gtctg | | |
| 60 W-I | | |
| ggtgaattcagtgtagtacaagagata | No | |
| gaaagaGATgtccttgctgaaagacaa | | |
| gtctg | | |
| 60 W-L | | |
| ggtgaattcagtgtagtacaagagata | Yes | Yes |
| gaaagaCAGgtccttgctgaaagacaa | | |
| gtctg | | |
| 60 W-M | | |
| ggtgaattcagtgtagtacaagagata | No | |
| gaaagaCATgtccttgctgaaagacaa | | |
| gtctg | | |
| 60 W-F | | |
| ggtgaattcagtgtagtacaagagata | Yes | No |
| gaaagaGAAgtccttgctgaaagacaa | | |
| gtctg | | |
| 60 W-P | | |
| ggtgaattcagtgtagtacaagagata | | |
| gaaagaGGGgtccttgctgaaagacaa | | |
| gtctg | | |
| 60 W-S | | |
| ggtgaattcagtgtagtacaagagata | No | |
| gaaagaGCTgtccttgctgaaagacaa | | |
| gtctg | | |
| 60 W-T | | |
| ggtgaattcagtgtagtacaagagata | No | |
| gaaagaGGTgtccttgctgaaagacaa | | |
| gtctg | | |
| 60 W-A | | |
| ggtgaattcagtgtagtacaagagata | No | |
| gaaagaGGCgtccttgctgaaagacaa | | |
| gtctg | | |
| 60 W-Y | | |
| ggtgaattcagtgtagtacaagagata | Yes | No |
| gaaagaGTAgtccttgctgaaagacaa | | |
| gtctg | | |
| 60 W-V | | |
| ggtgaattcagtgtagtacaagagata | Yes | Yes |
| gaaagaCACgtccttgctgaaagacaa | | |
| gtctg | | |
| 60 W-D | | |
| ggtgaattcagtgtagtacaagagata | No | |
| gaaagaGTCgtccttgctgaaagacaa | | |
| gtctg | | |
| 60 W-R | | |
| ggtgaattcagtgtagtacaagagata | No | |
| gaaagaCCGgtccttgctgaaagacaa | | |
| gtctg | | |
| 58k-G 59 D-G 60 W-G | | |
| ggtgaattcagtgtagtacaagagata | No | |
| gaaagaGCCCCCGCCgctgaaagacaa | | |
| gtctg | | |
| 59 D-P | | |
| ggtgaattcagtgtagtacaagagata | Yes | No |
| gaaagaccaGGGcttgctgaaagacaa | | |
| gtctg | | |
| 59 D-R | | |
| ggtgaattcagtgtagtacaagagata | Yes | No |
| gaaagaccaCCGcttgctgaaagacaa | | |
| gtctg | | |
| 59 D-A | | |
| ggtgaattcagtgtagtacaagagata | Yes | No |
| gaaagaccaGGCcttgctgaaagacaa | | |
| gtctg | | |
| 59 deleted so 58K fused to 60W | | |
| ggtgaaattcagtgtagtacaagagat | Yes | Yes |
| agaaagaccacttgctgaaagacaagt | | |
| ctg | | |
| 61 S-P | | |
| ggtgaattcagtgtagtacaagagata | Yes | No |
| gaaGGGccagtccttgctgaaagacaa | | |
| gtctg | | |
| 57 S-P | | |
| ggtgaattcagtgtagtacaagagata | Yes | No |
| gaaagaccagtccttGGGgaaagacaa | | |
| gtctg | | |
| insert G after amino acid 58 kGdw | | |
| ggtgaattcagtgtagtacaagagata | Yes | No |
| gaaagaccagtcGCCcttgctgaaaga | | |
| caagtctg | | |
| 58k-A 59 D-A 60 W-A | | |
| ggtgaattcagtgtagtacaagagata | No | |
| gaaagaGGCGGCGGCgctgaaagacaa | | |
| gtctg | | |

Modified MHC Molecules or Components Thereof for Use in Immunosuppression

There is a crystal structure of CD8alpha homodimer with HLA A2 (Gao et al Nature 387:630-634 1997) and a mouse crystal paper (Kern Immunity 9:519-530 1998). B2M residues 58-60 make contact with CD8, and there is evidence that mutagenesis of R4 in human CD8alpha/alpha or R8 in murine CD8 which both contact K58 in human and mouse class 1 alpha chains abolishes binding (Gilbin PNAS USA 91:1716-1720 and Kern Immunity 9:519-530 1998 respectively). Secondly, binding to the alpha 2 domain (where mutagenesis of Q115, D122 and E128 has been shown to abolish binding (Sun et al. JEM 182: 1275-1280 1995). Finally binding to the alpha 3 domain (where mutagenesis data has also highlighted the importance of residues 223-229 Salter nature 345:41-46 1990).

There is also evidence from murine CD4/class II that alpha chain mutants can block interaction (Konig et al. JEM 182:779-787 1995).

This is in addition to the beta chain mutants mentioned above (Konig nature 356:796-798 and Cammaota 799-801 1992). Thus, the invention includes mutation of both alpha and beta chains from class I and class II and the use of these modified molecules for general immune suppression. For selective and more specific immune suppression, peptides can be linked to to all four chains as discussed above.

Finally, the invention also provides the disruption or blocking of the interaction between the co-receptor and the MHC molecules by providing agents capable of blocking the interaction. These are preferably, blocking monoclonal antibodies to CD4, CD8, class I or class II or finally small molecule inhibitors/peptides to block the interactions.

Discussion

CD8⁺ CTL need to exercise potent cytotoxicity to eliminate foreign pathogens but at the same time they need to maintain unresponsiveness against self-antigens. Self-tolerance is achieved by both thymic (central) and post-thymic (peripheral) processes. Because central tolerance is incomplete, some potentially autoreactive T cells will escape to the periphery where tolerance can be achieved by several mechanisms including the induction of anergy or AICD upon exposure to their specific antigen. However in some cases this may be incomplete and the combination of genetic predisposition, injury, or exposure to cross-reactive antigens may activate these cells and cause autoimmunity. Current therapy for these conditions often relies upon broad spectrum immunosuppression with the consequent risk of opportunistic infection or malignancy. It would be desirable to manipulate the immune system to eliminate only the disease specific T cells. To achieve T cell tolerance as a therapeutic goal, it is essential to identify the self-antigens recognised by the T cells and to define means for specific targeting disease-triggering T cells. Peptide-based immunotherapies have been shown to work only in murine models where they can prevent diseases such as experimental autoimmune encephalomyelitis, a CD4⁺ T cell driven disease [Gaur, 1992] or virus-induced autoimmune diabetes mediated by CD8⁺ CTL [Aichele, 1996]. However, CTL deletion induced by the angonist peptide can result in severe immunopathological damage in both CD4 and CD8 driven murine models in vivo, such that its therapeutic utility is limited.

However, the inventors reveal herein a novel approach to delete antigen-specific CTL with minimal cellular activation. This new approach is exemplified by disrupting CD8 contact with the alpha three domain of the MHC class I/peptide complex. This approach is particularly useful for elimination of autoreactive or alloreactive CTL, for which in most human cases the antigenic peptides remain unidentified. In addition the data provides insights into the biological role of CD8/MHC class I interaction in regulation of the function and fate of CTL.

CD8 binding to MHC class I brings the intracellular domain into close proximity with the tyrosine kinase p56lck and other components of the TCR signalling complex. Blocking this interaction therefore reduces the avidity of TCR-ligand binding and prevents coreceptor-associated p56lck from joining the TCR/CD3 complex [Luescher, 1995]. Consistent with this, the inventors observed that blocking of CD8 substantially reduced phosphorylation of the TCR zeta chain which characterises the classical TCR signalling pathway in T cell activation and perforin-mediated killing of target cells. On the other hand, blocking of CD8 rendered T cells which were still fully competent to upregulate FasL and undergo AICD. Inhibition of CD8-associated p56lck by herbimycin A has also been shown to have little effect on FasL-mediated cytotoxicity (latinis-km, blood 96 87/871). Thus, it appears that very limited TCR signalling can trigger surface expression of FasL conceivably by translocation of preformed, intracellular FasL, to the cell surface as previously described in other cell types [Kiener, 1997; Lowin, 1996].

The CD8-independent death of CTL has several implications: firstly surface expression of CD8 could regulate target cell and CTL death. Indeed downregulation of CD8 has been demonstrated to occur on stimulation of some CTL clones [Robbins, 1991]. The inventors results suggest that this will reduce target cell killing without affecting CTL apoptosis thus downregulation of CD8 provides a potent mechanism to generate peripheral tolerance. Further analysis of the CTL assay presented in FIG. 1a shows that following antigen contact CTL, which are initially CD8 high, separate into two populations: CD8 high and CD8 low. Most of the apoptotic cells (80%) were CD8 low. The inventors propose that the initial contact of CTL with targets leads to full T cell activation, killing of the target and downregulation of CD8. At this stage the reduction of CD8 expression may limit target cell killing whilst leaving CTL apoptosis unaffected. This, combined with the downregulation of the T cell receptor, will limit the ability of CTL to engage and kill multiple targets and may form the basis of immunological exhaustion. Support for this hypothesis comes from a recent report showing that inhibition of CD8 gene expression by methylation led to the death of misselected peripheral CD8 T cells in vivo via a Fas/FasL pathway [Pestano, 1999].

In summary the inventors findings provide a mechanism for the deletion of specific CTL populations, without the concomitant T cell activation and end organ damage that can be induced by full stimulation with agonist peptide [Combadiere, 1998]. Peptide/MHC complexes created with mutant heavy chain lacking CD8 interaction would not lead to this detrimental T cell activation and may allow antigen specific deletion of the disease-mediating CTL. The other advantage of this approach is that it can allow deletion of CTL without the need to identify the antigenic peptide. So for instance in these experiments the inventors were able to delete B4402-allospecific CTL by expressing an alpha 3 domain mutant in cells where it is loaded with a variety of endogenous peptides. It would also be possible to produce soluble forms of these molecules loaded with unknown host peptides by expression in mammalian cells and cells derived from the patient for whom therapy was planned.

1. Use of an immunocomplex comprising (a) an MHC molecule or functional derivative or fragment thereof having a modified co-receptor binding domain such that T cell co-receptor interaction is prevented, and (b) a peptide antigen, in the preparation of a medicament for treating a patient requiring selective suppression of the immune system.

2. Use according to claim 1 wherein the patient has an autoimmune disease.

3. Use according to claim 1 wherein the patient is the recipient of a transplanted tissue.

4. Use according to any one of claims 1 to 3 wherein said MHC molecule was a MHC class I molecule.

5. Use according to claim 4 wherein the co-receptor CD8.

6. Use according to any one of claims 1 to 3 wherein the MHC molecule is a MHC class II molecule.

7. Use according to claim 6 wherein the co-receptor is CD4.

8. Use according to any one of claims 1 to 5 wherein the MHC molecule is modified in the α3 domain of the heavy chain.

9. Use according to claim 8 wherein said modification is by addition, substitution or deletion of one or more amino acids native to the α3 or α3 domain.

10. Use according to any one of claims 1 to 3 wherein the fragment of the MHC molecule is the β2M component.

11. Use according to claim 10 wherein the β2M component is modified at any one or more of amino acid residues 57 to 61.

12. Use according to claim 11 where in the β2M component is modified at any one or more amino acid residues 58 to 60.

13. Use according to any one of claims 10 to 12 wherein the modified β2M is selected from the group provided in Table 1.

14. Use according to any one of claims 10 to 13 wherein the β2M component is modified at least at residue 60.

15. Use according to any one of claims 10 to 13 wherein the β2M component is modified at least at residue 58.

16. A method of producing an immunocomplex comprising (a) an MHC molecule or functional derivative or fragment thereof having a modified co-receptor binding domain such that T cell co-receptor interaction is prevented, and (b) a peptide antigen, said immunocomplex being suitable for treating a patient requiring selective immune suppression, said method comprising the steps of
(a) obtaining donor cells associated with eliciting a immune response requiring suppression;
(b) determining the MHC allele expressed by said donor cells;
(c) manipulating said donor cells to express a modified MHC allele, said modified MHC lacking the ability to bind co-receptor but maintaining the ability to present peptide antigen as an immunocomplex; and
(d) isolating said immunocomplex.

17. A method according to claim 16 wherein said immunocomplex is isolated from said donor cells.

18. A method according to claim 16 or claim 17 wherein the donor cells are from a patient suffering from an autoimmune disease.

19. A method according to claim 16 or claim 17 wherein the donor cells are from a donor tissue awaiting transplantation.

20. A method according to any one of claims 16 to 19 wherein the manipulation of the donor cells was caused by site directed mutagenesis of the nucleic acid encoding the MHC molecule.

21. A method according to claim 20 wherein the MHC was a MHC class I molecule and the mutagenesis was directed to the α3 or the α2 domain of the heavy chain.

22. A method according to any one of claims 16 to 19 wherein manipulation of the donor cells includes disruption of the MHC loci so that native MHC production is prevented and introduction of a vector encoding a modified MHC molecule of the same allele as the native MHC molecule.

23. A method according to any one of claims 16 to 22 further comprising the step of purifying said immunocomplex ready for administration to a patient.

24. A method according to claim 23 further comprising the step of producing a pharmaceutical composition comprising said immunocomplex.

25. Use of an immunocomplex produced by a method according to any one of claims 16 to 24 in the preparation of a medicament for treating a patient requiring selective immune suppression.

26. A method of producing an immunocomplex comprising (a) an MHC molecule or functional derivative or fragment thereof having a modified co-receptor binding domain such that T cell co-receptor interaction is prevented, and (b) a peptide antigen, said immunocomplex being suitable for treating a patient requiring selective immune suppression, said method comprising the steps of
(a) transfecting a cell with a vector encoding a MHC molecule having a modified co-receptor binding domain such that T cell co-receptor interaction is prevented;
(b) expressing said modified MHC molecule in said cell; and
(c) isolating and purifying said immunocomplex.

27. A method according to claim 26 wherein the MHC molecule is a MHC class I molecule and the co-receptor is CD8.

28. A method according to claim 26 wherein the MHC molecule is a MHC class II molecule and the co-receptor is CD4.

29. A method according to any one of claims 26 to 28 wherein the cell is an MHC negative cell.

30. A method according to any one of claims 26 to 29 wherein the cell is derived from a tissue associated with an autoimmune disease.

31. A method according to any one of claim 26 to 29 wherein the cell is derived from a organ to be transplanted.

32. Use of a modified MHC molecule or functional derivative or fragment thereof in the preparation of a medicament for suppressing the immune system in a patient, said MHC molecule or functional derivative or fragment thereof being modified in the co-receptor binding domain such that co-receptor interaction is prevented.

33. Use according to claim 32 wherein the MHC molecule is a class I molecule, and the modification is in the α3 or α2 domain of the heavy chain.

34. Use according to claim 32 wherein the MHC molecule is a class II molecule and the modification is in the α domain or the β-chain β domain.

35. Use according to claim 32 wherein the MHC molecule fragment is the β2M component.

36. Use according to claim 35 wherein the β2M component is modified in any one or more of residues 57 to 61.

37. Use according to claim 36 wherein the β2M component is modified in any one or more of residues 58 to 60.

38. Use according to any one of claims 35 to 37 wherein the β2M component is modified at least at residue 60.

39. Use according to any one of claims 35 to 38 wherein the β2M component is modified at least at residue 58.

40. Use according to any one of claims 35 to 38 wherein the modified β2M is selected from the group provided in Table 1.

41. A method of treating a patient requiring selective immune suppression, said method comprising the steps of
(a) determining the MHC allele associated with eliciting the immune response requiring suppression;
(b) producing an immunocomplex comprising the MHC allele or functional derivative or fragment thereof having a modified co-receptor binding domain, and a peptide antigen; and
(c) administering said immunocomplex to said patient in order to selectively suppress the immune response.

42. A method according to claim 41 wherein the immunocomplex administered to the patient is nucleic acid encoding the modified MHC allele or functional derivative or fragment thereof, and the peptide antigen.

43. A method according to claim 41 wherein the immunocomplex administered to the patient is a protein comprising modified MHC allele or functional derivative or fragment thereof and peptide antigen.

44. An immunocomplex comprising an MHC molecule or functional fragment thereof having a modified co-receptor binding domain such that co-receptor interaction is prevented, and a peptide antigen.

45. An immunocomplex according to claim 44 wherein the functional fragment is β2M.

46. A nucleic acid sequence encoding an immunocomplex according to 44 or claim 45.

47. A nucleic acid vector comprising the nucleic acid sequence according to claim 46.

48. A pharmaceutical composition comprising an immunocomplex according to claim 44 or claim 45 or a nucleic acid sequence according to claim 46 or 47, and a pharmaceutically acceptable carrier.
